(19) United States
(12) Patent Application Publication
(10) Pub. No.: US 2024/0148717 A1
(43) Pub. Date: May 9, 2024

(54) COMBINATION THERAPY FOR TREATMENT OF CONDITIONS ASSOCIATED WITH AGING

(71) Applicant: Carlo RATTI, Cambridge, MA (US)

(72) Inventor: Carlo RATTI, Cambridge, MA (US)

(21) Appl. No.: 18/548,697

(22) PCT Filed: Mar. 1, 2022
(86) PCT No.: PCT/US2022/018382

§ 371 Date: Sep. 1, 2023

Related U.S. Application Data
(60) Provisional application No. 63/154,902, filed on Mar. 1, 2021.
(60) Provisional application No. 63/155,006, filed on Mar. 1, 2021.

(51) Int. Cl. A61K 31/4706; A61K 31/015; A61K 31/047; A61K 31/05; A61K 31/12; A61K 31/255; A61K 31/65; A61K 31/7052; A61K 38/48; A61P 29/00

(52) U.S. Cl. CPC A61K 31/4706; A61K 31/015; A61K 31/047; A61K 31/05; A61K 31/12; A61K 31/255; A61K 31/65; A61K 31/7052; A61K 38/482; A61P 29/00

(57) ABSTRACT

Inflammatory disease and signs of aging, such as graying of hair can be treated by use of a microbiofilm-inhibiting, destroying or disrupting agent is administered especially when used in in combination with an antimicrobial agent. Useful said microbiofilm-inhibiting, destroying or disrupting agents include Chloroquine or Hydroxychloroquine. Nattokinase, Curcumin or a Cannabinoids and allicin. Useful antimicrobials include Penicillin (in particular Penicillin G), beta-lactams (ceftriaxone), erythromycin, tetracycline, doxycycline and chloramphenicol. For example, those having antioxidant properties such as azithromycin, doxycycline or antioxidant.

### CROSS REFERENCE TO RELATED APPLICATIONS

The present application claims priority from U.S. provisional applications 63/154,902 and 63/155,006 both of which were filed on 1 Mar. 2021. The contents of both of these provisional applications are incorporated herein in their entirety.

### FIELD OF THE INVENTION

The present invention relates to a combination therapy for treatment of conditions associated with aging—including graying of hair other autoimmune and chronic inflammatory diseases.

### BACKGROUND OF THE INVENTION

Graying of the hair is a common sign of aging in humans. It occurs when the light-absorbing melanin pigments produced from cells called melanocytes are being lost [1]. Melanocytes are derived from melanocyte stem cells (MeSCs), which are located in a part of the hair follicle

Recently, the accumulation of hydrogen peroxide (H₂O₂) and more generally Reactive Oxygen Species (ROS) has been linked graying of hair—including hair follicle melanocyte apoptosis and DNA damage [2]. This accumulation has been attributed to the loss of hydrogen-peroxide-reducing enzyme catalase and methionine sulfoxide repair mechanisms [2]. However, hair follicles are also colonized by microorganisms [3] and ROS play a major function in host-microbial interactions [4].

More generally, the human skin is colonized by a diverse community of microorganisms that are essential for maintaining healthy skin [3, 5]. Many skin microbes are commensals and play a role in pathogen defense and modulation of the human immune system. Recent studies have suggested that certain common skin bacteria may contribute to protecting the host against oxidative stress [6, 7]. However, the microbiome has also been associated with skin diseases. For example, certain bacteria such as Streptococcus pneumoniae release ROS to inhibit immune responses and enable pathogenic colonization of the host [4]. Lactobacillus species are also known to produce ROS and have thus been considered as a nonspecific antimicrobial defense mechanism (especially in the vaginal ecosystem, see for instance [8]).

We have discovered that microbial communities organized in biofilms—and covered by a shield made of host Z-DNA and a Extracellular Polymeric Substances (EPS) and able to communicate through electrical and chemical signaling based on quorum sensing—play a key role in the graying of hair and in other autoimmune and inflammatory diseases through ROS production and the formation of plaques. Hence, the degradation and dispersal of biofilms, together with the use of antiplaque, antimicrobial and antibiotics can help reverse the graying of hair—and that of other autoimmune (such as Lupus, chronic arthritis, asthma, IBD, etc.) and chronic inflammatory diseases (such as arteriosclerosis, prostatitis, Alzheimer Disease, Parkinson Disease, etc.).

### SUMMARY OF THE INVENTION

The present invention addresses the phenomena noted above by combining agents that allow the removal of microbial biofilms and plaques. This effect can be implemented in different ways to treat different conditions. For example, inflammatory disease and signs of aging, such as graying of hair can be treated by use of a microbiofilm-inhibiting, destroying or disrupting agent is administered especially when used in in combination with an antimicrobial agent.

The present invention provides treatment for many diseases, which are based on inflammation and the possible formation of plaques, including—but not limited to—atherosclerosis, prostatitis, Alzheimer's disease, Parkinson disease, chronic arthritis, Lupus, Multiple Sclerosis, asthma, IBD— as well as auto-immune diseases such as Lupus, Multiple Sclerosis, psoriasis—by use of a combined protocol comprised of:
An agent to degrade host the shield made of Z-DNA into B-DNA, as Chloroquine or Hydroxychloroquine.
Biofilm dispersal agents that interfere with quorum sensing such as Nattokinase. Curcumin or Cannabinoids in order to reduce bacteria to their planktonic form
Plaque-removal agents such as Allicin.
Antibacterials chosen to be effective against the various communities contained into the biofilms in question.

### BRIEF DESCRIPTION OF THE FIGURES

FIG. 1 shows Patient A's uBiome at the end of February 2019.

FIG. 2 shows use of PICC for administration of Penicillin G.

FIG. 3 shows plaque healing for Patient A between 22 Aug. 2019 and 23 Sep. 2019.

FIG. 4 shows part of Patient A's right hand during treatment.

FIG. 5 shows Patient A's left hand during treatment.

FIG. 6 shows skin of Patient A's left hand on Sep. 7, 2019 and Sep. 22, 2019.

FIG. 7 shows partial reversing of facial gray hair at the end of treatment described below.

### DETAILED DESCRIPTION OF THE INVENTION

Suitable antibacterial compounds for use in the invention depend upon the nature of the target bacteria, but for many conditions one of the following types of compound, and in many cases combinations of antibacterials from each of these groups may be used:
A compound active against spirochetes, such as Penicillin (in particular Penicillin G), beta-lactams (ceftriaxone), erythromycin, tetracycline, doxycycline or chloramphenicol.
Anti-chlamydial compound, typically one having antioxidant properties, such as azithromycin, doxycycline or antioxidant carotenoid such as Lutein and Lycopene (cf. recent work by Zigangirova et al., 2017 [9].

Such combinations are particularly useful in treating conditions where the initial biofilm converts into a plaque as the diseased progresses such as Alzheimer's disease.

The precise dosage used for each of these materials will depend on the actual compounds used. However in one embodiment a dose of from 18-30, for example 21-28 million units per day of penicillin G for a period of from 4 to 12 weeks, for example about 8 weeks, is combined with a twice daily dose of lycopene of from 10-30 mg per dose and allicin powder administered in doses of from 1-10 grams per day.

In other situations, a selection of antibacterials based on the results of shotgun metagenomic analysis to eliminate bacteria associated with peroxide accumulation, such as rifampin, azithromycin and doxycycline or a combination of any two of these or of all three may be used. Such combinations are particularly useful in slowing the graying of hair.

In other situations, specific antimicrobials can be used based on phage or CRISPR (clustered regularly interspaced short palindrome repeats).

Formulations of the compositions of the present invention will depend on the particular use to which they are intended. For example, if the objective is to prevent or minimize the growth of plaques in the brain, it is necessary for the composition to be able to pass through the blood-brain barrier.

In order to penetrate the blood-brain barrier, the anti-spirochete compound such as penicillin may be administered using a peripherally inserted central catheter (PICC), for example a portable PICC in which intravenous administration is effected into the patient's arm. Suitably, administration following the protocol for neuro-syphilis or neuro-borreliosis is used (Aqueous crystalline penicillin G 18-24 million units per day, administered as 3-4 million units IV every 4 hours or continuous infusion, CDC 2019 [10]).

Lycopene is present in a number of fruits and vegetables, including tomatoes and derivatives thereof such as ketchup and tomato paste. In the present invention, it is, however, preferably used in tablet form.

Biofilms and plaque-removal agents include Allicin, an amino acidic naturally occurring in garlic. In addition to its antimicrobial properties, which have been thoroughly studied (see for instance Cutler and Wilson, 2004 [11]), allicin has a very strong plaque-removing effect as shown by Gonen et al., 2005 and Kumar, 2019 [13]. Preferably, however, the biofilm and plaque removal agent is administered as a powder. Other plaque removal or plaque-control agents include statins such as Atorvastatin. The above process can be sped up through vasodilation (for instance through the use of nitrites and/or exposure to sunlight, Allen and Gow, 2009 [14], Holliman et al., 2017 [15]).

Other agents helping with biofilms degradation are Chloroquine and Hydroxychloroquine, which convert host Z-DNA used as shield by biofilms into B-DNA— hence, making it susceptible to host immune system attack. Curcumin and cannabinoids also have a well-documented anti-biofilm properties as they interfere with quorum sensing.

In situations such as that where the objective is to slow or stop the graying of hair. The combination, for example of an anti-biofilm agent and an antimicrobial is formulated for topical administration, for example as a cream or lotion and may, for example be incorporated in hair care products such as hair conditioners.

1. Case study—Patient A

A 48 y.o patient (below, “Patient A”) was hospitalized on 10 Jun. 2019 and reported the following symptoms:
Fatigue;
Rash similar to a syphilis rash Patient A experienced 6 months previous;
Blurred vision; and
Headache and impaired thinking.

Initial tests came out as follows:
CSF White blood cell (WBC) count was 30, suggesting infection or inflammation of the central nervous system.
VDRL in blood 1:512.
VDRL in spinal tab also positive.

Based on the above results Patient A was treated with aqueous crystalline penicillin G 24 million units per day, administered intravenously every 4 hours. Therapy seemed effective, as after a few days Patient A started feeling better with vision improving.

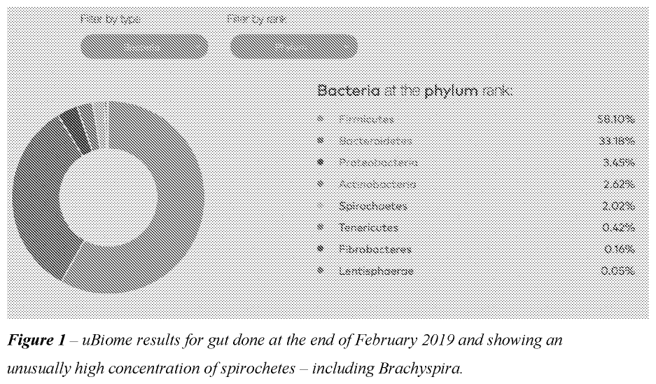

However, new medical results came in during the following days which questioned the initial diagnosis, but which indicated that the condition neuro-spirochetosis. On 23 Feb. 2019 Patient A had performed a full microbiome test via start up uBiome, which showed an unusually high concentration of spirochetes (See FIG. 1). Full analysis of prevalent bacteria did not show Treponemas or Borrelias. However, other spirochetes were present which could create systemic infection. Among these bacteria, Patient A had Brachyspira, a slow-growing anaerobic spirochete that can colonize the large intestine of animals including humans. While Brachyspira is known for causing diarrhea, it was recently discovered that it could also attack other organs. It was found in the Alzheimer's brain (Miklossy, 2011 [16]) and has been recently been associated with other human chronic conditions (Hampson, 2017 [17]). One human experimental infection was recently performed, where a volunteer drank cultures of Brachyspira isolated from an Australian Aboriginal child with diarrhea, and reported headaches, among other symptoms. (Hampson, 2017 [18]).

After a number of medical consultations, Patient A agreed to follow a non-standard protocol, extending penicillin treatment s to a 6+week course for neuro-spirochetosis. On 19 August Patient A was dismissed from hospital but continued with penicillin via PICC (aqueous crystalline penicillin G 24 million units per day continuous infusion).

In addition to penicillin, Patient A continued taking Lycopene supplements to cure a pre-existing Chlamydia Pneumoniae infection.

In addition, on 22 August Patient A added a plaque removal agent. As there are no reported interactions between Penicillin and Allicin, Allicin supplements were chosen (10×450 mg daily lab-grade allicin powder—and then raised it to 50×450 mg daily).

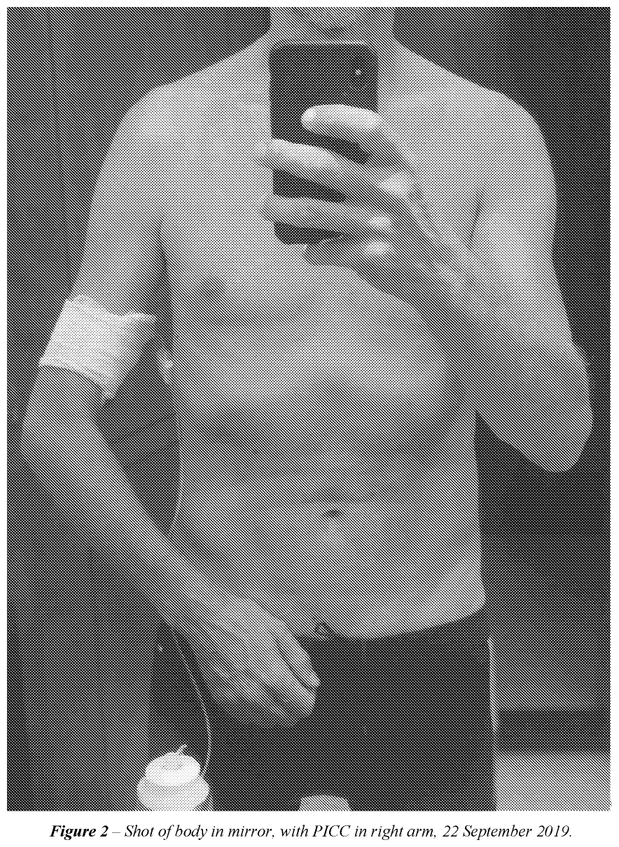

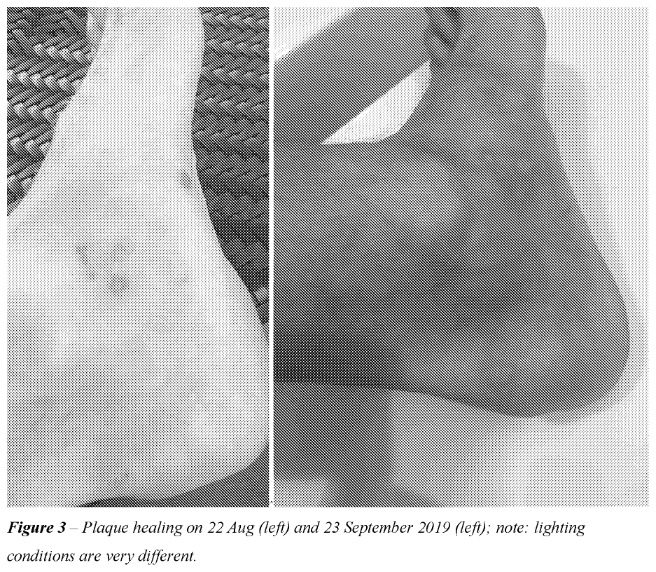

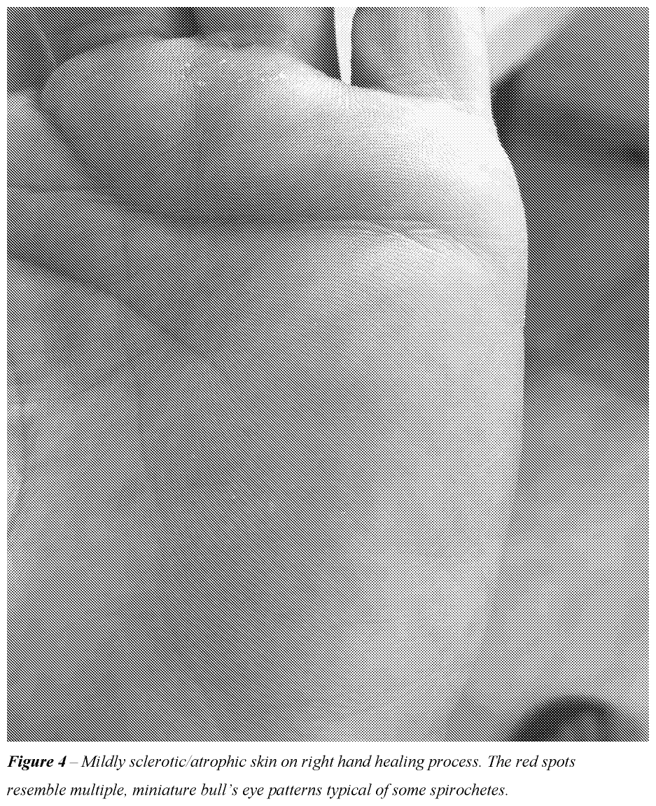

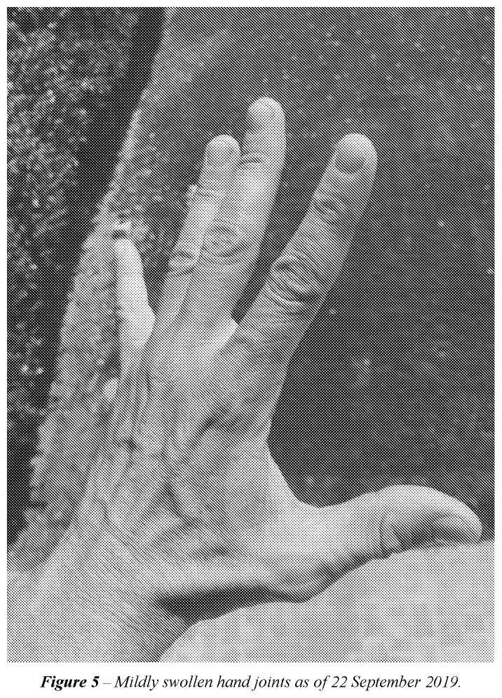

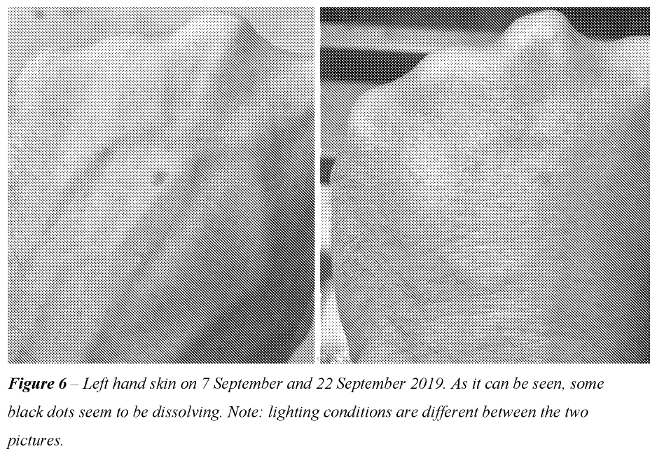

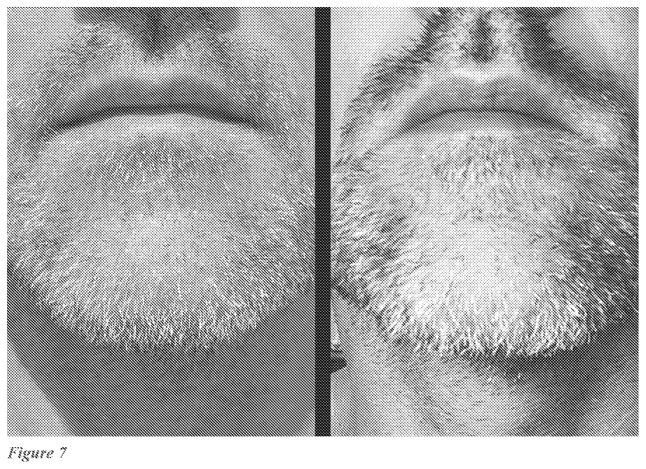

Patient reported the following improvements—including the *very surprising* disappearance of minor symptoms he had not been aware of beforehand. A summary follows:
Eyes. Vision blur resolved almost completely.
Brain. Improved cognitive functions short-and-long-term memory (he reported faster typing).
Numbness. Some minor parts of his body that had been numb since the 2008 neuropathy, such as the tips of the toes, started to regain sensitivity.
Body. Even in a condition of little exercise while being hospitalized the patient lost about 5 kg in 2 weeks and seemed more toned and in generally better condition (FIG. 2).
Asthma. Patient A reported better breathing. A couple of times when he was around 18 and 35 year two separate doctors suggested that he should be checked for asthma—although he never completed the investigations.
IBD. Bowel movement, which beforehand suggested sub-clinical IBD, became normal.
Arthritis. Joints in hands started swelling, and then healing (see FIG. 5). Arthritis has been reported both with spirochetes and Chlamydia Pneumoniae infections (Steere, 1987 [19], Porritt, 2016 [20]).
Prostate. Patient A never had prostate problems. However, he reported a larger urine flow and more frequent nocturnal penile tumescence.
Skin. Lesions and sclerotic plaques on his hands, which dated back from 2008, started healing. They resembled a sub-clinical manifestation of Acrodermatitis Chronica Atrophicans, which appears in phase 3 of European borreliosis (see FIG. 3). Incidentally, the healing process highlighted a small-scale bull's eye patterns, with a white patch and a red dot at the center (FIG. 4). Among the affected parts, there was also his penis' skin, which looked similar to the Borrelial Lymphocytoma Cutis reported by Kandhari, 2014 and which started healing.
White and black dots on skin. Several black and white dots on hands arms and legs—very common with age and exposure to sun—started dissolving (see FIG. 6). The process seemed to accelerate with sun exposure.
At the end of therapy patient also added hydroxychloroquine 200 mg per day for one months and high dosage curcumin (3 g per day), which resulted in partial reversal of gray hair (see FIG. 7).

### REFERENCES

1. Nishimura, E. K., Granter, S. R., and Fisher, D. E. (2005). Mechanisms of Hair Graying: Incomplete Melanocyte Stem Cell Maintenance in the Niche. Science 307, 720-724.
2. Wood, I. M., Decker, H, Hartmann, H, Chavan, B., Rokos, H, Spencer, J. D., Hasse, S., Thornton, MI, Shalbaf M, Paus, R., et al. (2009). Senile hair graying: H2O2-mediated oxidative stress affects human hair color by blunting methionine sulfoxide repair. The FASEB Journal 23, 2065-2075.
3. Nakatsuji, T, Chiang, H-1., Jiang, S B., Nagarajan, H., Zengler, K, and Gallo, R. L. (2013). The microbiome extends to subepidermal compartments of normal skin. Nature Communications 4, 1431
4. Erttmann, S. F., and Gekara, N. O. (2019). Hydrogen peroxide release by bacteria suppresses inflammasome-dependent innate immunity. Nature Communications 10, 3493.
5. Byrd, A. L., Belkaid, Y., and Segre, J. A. (2018). The human skin microbiome. Nature Reviews Microbiology 16, 143-155.
6. Andersson, T, Ertiirk Bergdahl, G., Saleh, K., MagntisdOttir, H, Stodkilde, K., Andersen, C. B. F., Lundqvist, K, Jensen, A., Briiggemann, H, and Lood, R. (2019). Common skin bacteria protect their host from oxidative stress through secreted antioxidant RoxP. Scientific Reports 9, 3596.
7. Allhorn, M, Arve, S., Briiggemann, H, and Lood, R. (2016). A novel enzyme with antioxidant capacity produced by the ubiquitous skin colonizer Propionibacterium acnes. Scientific Reports 6, 36412.
8. Eschenbach, D. A., Davick, P. R., Williams, B. L., Klebanoff, S. J, Young-Smith, K., Critchlow, C. M, & Holmes, K K (1989). Prevalence of hydrogen peroxide producing Lactobacillus species in normal women and women with bacterial vaginosis. Journal of clinical microbiology, 27(2), 251-256.
9. Zigangi. ova N A, Morgunova E Y, Fedina E D, Shevyagnia N J/Borovaya I G, Zhukhovitsky V G, Kyle N H. Peiyaev I M Lycopene Inhibits Propagation of Chlamydia Infection. Scientifica (Cairo), 2017; 2017: 1478625, doi: 10.1155/2017/1478625. Epub 2017 Aug. 29. PubMed PMID: 28948060; PubMed Central PMCID: PMC5602621
10. https://www.cdc.gov/std/tg2015/syphilis.htm #Neurosyphilis
11. Cutler R R, Wilson P. Antibacterial activity of a new, stable, aqueous extract of allicin against methicillin-resistant Staphylococcus aureus. Br J Biomed Sci. 2004; 61(2): 71-4. PubMed PMID: 15250668,
12. Gonen A, Harats D, Rabinkov A, Miron T, Mirelman D, Wilchek M, Weiner L, Ulman E, Levkovitz H, Ben-Shushan D, Shaish A. The antiatherogenic effect of allicin: possible mode of action. Pathobiology. 2005; 72(6): 325-34. doi: 10.1159/000091330. PubMed PMID: 16582584.
13. Kumar S, Kumar S, Ram H Anti Aggregation Property of Allicin by In Vitro and Molecular Docking Studies. J Exp Nurosci. 2019; 13: 1179069519866185. doi: 10.177/1179069519866185. eCollection 2019. PubMed PMID: 31384132; PubMed Central PMCID: PMC6664621.
14. Allen J D, Gow A I Nitrite, N O and hypoxic vasodilation. Br J Pharmacol. 2009 December; 158(7): 1653-4. doi: 10.1111/j. 1476-5381.2009.00447.x. PubMed PMID: 19888966; PubMed Central PMCID: PMC2801205.
15. Holliman G, Lowe D, Cohen H, Felton S, Raj K Ultraviolet Radiation Induced Production of Nitric Oxide: A multi-cell and multi-donor analysis. Sci Rep. 2017 Sep. 11; 7(1): 11105. doi: 10. 1038/s41598-017-11567-5. PubMed PMID: 28894213; PubMed Central PMCID: PMC5593895.
16. Miklassy J Alzheimer's disease—a neurospirochetosis. Analysis of the evidence following Koch's and Hill's criteria. Jr Neuroinflamniation. 2011 Aug. 4; 8:90.410 10.1186/1742-2094-840. Review. PubMed PMID: 21816039; PubMed Central PMCID: PIVIC 3171359,
17. Hampson D J. The Spirochete Brachyspira pilosicoli, Enteric Pathogen ofAnimals and Humans. Clin Microbiol Rev. 2018 January; 31(1). doi: 10. 1128/CMR.00087-17. Print 2018 Jan. Review. PubMed PMID: 29187397; PubMed Central PMCID: PMC5740978.
18. Hampson D J. The Spirochete Brachyspira pilosicoli, Enteric Pathogen ofAnimals and Humans. Clin Microbiol Rev. 2018 January; 31(1). doi: 10.1128/CMR. 00087-17. Print 2018 Jan. Review. PubMed PMID: 29187397; PubMed Central PMCID: PMC5740978.
19. Steere A C, Schoen R T, Taylor E. The clinical evolution of Lyme arthritis. Ann Intern Med. 1987 November; 107(5):725-31. doi: 10.7326/0003-4819-107-5-725. PubMed PMID: 3662285.
20. Porritt R A, Crother T R. Chlamydia pneumoniae Infection and Inflammatory Diseases. For Immunopathol Dis Therap. 2016; 7(3-4): 237-254. doi: 10.1615/ForumImmunDisTher.2017020161. PubMed PMID: 30687565; PubMed Central PMCID: PMC6345537.
21. Kandhari R, Kandhari S, Jain S. Borrelial lymphocytoma cutis: a diagnostic dilemma. Indian J Dermatol. 2014 November; 59(6): 595-7. doi: 10.4103/0019-5154.143530. PubMed PMID: 25484392; PubMed Central PMCID: PMC4248499

1. A method of treatment of a diseases based on chronic inflammation and the possible formation of plaques which comprises administering a microbial biofilms-inhibiting, destroying or disrupting agent to a patient in need thereof.

2. A method as claimed in claim 1, wherein said microbiofilm-inhibiting, destroying or disrupting agent is administered in combination with an antimicrobial agent.

3. A method of treatment of diseases which are based on inflammation and the possible formation of plaques as claimed in claim 1, which comprises administering to a patient in need thereof:
At least one agent to degrade host the shield made of Z-DNA into B-DNA;
At least one biofilm dispersal agent that interfere with quorum in order to reduce bacteria to their planktonic form;
At least one plaque-removal agent; and
At least one antimicrobial chosen to be effective against the various communities contained into the biofilms in question.

4. A method as claimed in claim 3, wherein said agent to degrade said host shield is as Chloroquine or Hydroxychloroquine.

5. A method as claimed in claim 3, wherein said biofilm dispersal agent is Nattokinase, Curcumin or a Cannabinoids.

6. A method as claimed in claim 3, wherein said plaque removal agent is allicin.

7. A method as claimed in claim 3, wherein said antibacterial agent is a compound active against spirochetes.

8. A method as claimed in claim 7, wherein said ant anti bacterial agent is selected from the group consisting of Penicillin (in particular Penicillin G), beta-lactams (ceftriaxone), erythromycin, tetracycline, doxycycline and chloramphenicol.

9. A method as claimed in claim 3, wherein said anti-microbial agent is an anti-chlamydial compound, having antioxidant properties.

10. A method as claimed in claim 9, wherein said compound is azithromycin, doxy cy dine or antioxidant carotenoid.

11. A method as claimed in claim 10, wherein said compound is lutein or lycopene or a combination thereof.

12. A method as claimed in claim 3, which comprises administering from 18-30, for example 21-28 million units per day of penicillin G for a period of from 4 to 12 weeks.

13. A method as claimed in claim 3, which comprises administering a twice daily dose of lycopene of from 10-30 mg per dose and allicin powder administered in doses of from 1-10 grams per day.

14. A method as claimed in claim 12, which comprises administering a twice daily dose of lycopene of from 10-30 mg per dose and allicin powder administered in doses of from 1-10 grams per day.

15. A method of slowing the graying of hair and other signs of aging which comprises administering a microbial biofilms inhibiting, destroying or disrupting agent to a subject in need thereof.

16. A method as claimed in claim 15, wherein said microbiofilm-inhibiting, destroying or disrupting agent is administered in combination with an antimicrobial agent.

17. A method as claimed in claim 15 or 16, wherein said microbiofilm-inhibiting, destroying or disrupting agent is allicin.
